(19) United States
(12) Patent Application Publication
(10) Pub. No.: US 2019/0295697 A1
(43) Pub. Date: Sep. 26, 2019

(54) Payor Facilitation and Health Related Outcomes Tracker

(71) Applicant: Jawad T. Ali, Austin, TX (US)

(72) Inventor: Jawad T. Ali, Austin, TX (US)

(21) Appl. No.: 16/357,611

(22) Filed: Mar. 19, 2019

Related U.S. Application Data
(60) Provisional application No. 62/645,654, filed on Mar. 20, 2018.

(51) Int. Cl. G16H 10/60; G06Q 40/08

(52) U.S. Cl. CPC G16H 10/60; G06Q 40/08

(57) ABSTRACT

The present invention is a computer-based payor facilitation and health related outcomes tracker that allows for upload of insurance related documentation as well as track their progress. The payor facilitation and health related outcomes tracker includes the creation of a patient's computer-based user account, the enablement of the chat window, the creation of patient personalized progress tracking as well as a computer-based document guidance, uploading and sending function.

### CROSS-REFERENCE TO RELATED APPLICATIONS

The present application claims benefit from a U.S. provisional application No. 62/645,654 filed on Mar. 20, 2018. The entire disclosure of the application is hereby incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC

Not Applicable

### FIELD OF INVENTION

The present invention combines investing the patient in the payor approval facilitation process with patient care guidance and patient-reported outcomes tracking. It relates to facilitating insurance pre-approval by providing document uploading and application progress updates.

The objective of the present invention is to have a computer-based application that allows for upload of insurance-related documentation as well as tracking their progress. It also combines insurance approval functionality with patient-reported questionnaires and outcomes tracking.

### BACKGROUND OF THE INVENTION

Patients who are candidates for medical drugs or devices often face difficulties in obtaining insurance approval. Hospitals may require pre-approval for novel devices and drugs or those with uncertain insurance reimbursement. In this case, the healthcare provider and/or medical office must submit a detailed application to the payor or third-party facilitators.

This detailed patient file usually contains, but is not limited to, patient symptoms, test results and data about the drug or device. This patient file could take weeks or even months to get reviewed and obtain a response from the payor. Also, patients are usually in the dark during this process and are uninformed about the progress or status of their drug or device approval. This causes patients to become frustrated and the patients often feel abandoned and ignored by their healthcare provider.

Unfortunately, a reviewed patient file may still get rejected by the payor. If the reviewed patient file is rejected, the medical office must submit additional documents in a resubmission patient file. There is often a delay in the patient finding out about the rejection as well as a further delay in submitting the resubmission patient file. Consequently, many patients who are potential candidates for drugs or devices requiring payor pre-approval abandon seeking the desired drug or device during the approval process. This causes a significant decrease in the number of patients using these products and also getting the best medical care.

This process leaves medical providers and patients very dissatisfied and it also causes decreased sales for companies selling these products. Patients, providers, third-party facilitators and payors would greatly benefit from a method that allows patients to be informed about the progress of their patient file thereby keeping them invested throughout this tedious process. In addition, healthcare providers as well as medical technology companies, would benefit immensely from patients' feedback about their symptoms and experience throughout the process. The feedback would also help keep the patients informed and invested thought the process.

Currently, no application utilizes a computer-based application to upload health insurance documentation while also tracking the progress of the application for that patient. While there are applications for perioperative guidance and symptom tracking, none combine this with the insurance application function. Furthermore, most applications function for limited periods of time and none achieve this function for the long term.

Because of the above mentioned problems, it is desirable to have a computer-based application that reduces patient file review times by streamlining the application process while also decreasing patient dropout rates by keeping the patient involved and informed about their progress in the approval process and to let them know which stage they are in the process. In addition, it is important for healthcare providers and companies to know how patients are doing so they limit adverse events and improve their product as well as healthcare delivery.

### BRIEF SUMMARY OF THE INVENTION

One example embodiment of the present invention provides a method that includes the creation of a patient's computer-based user account, the enablement of the chat window, the creation of patient personalized progress tracking as well as a computer-based document guidance, uploading and sending function.

Another example embodiment of the present invention provides a method that includes the creation of a patient's computer-based user account, the enablement of the chat window and the creation of patient personalized progress tracking, a computer-based document guidance, uploading and sending function as well as computer-based guidance through the pre-device or drug, peri-device or drug and post-device or drug phase.

Another example embodiment of the present invention provides a method that includes the creation of a patient's computer-based user account, the enablement of the chat window and the creation of personalized progress tracking, a computer-based guidance for required documents, a computer based uploading and sending function, a computer-based guidance through the pre-device or drug, peri-device or drug and post-device or drug phase that also contains a patient questionnaire that alerts the provider about concerning symptoms while also providing useful data about the efficacy of the drug or device.

### BRIEF DESCRIPTION OF THE DRAWINGS

FIG. 1 illustrates multiple components that the computer-based application may include.

FIG. 2A illustrates a flow diagram of an example embodiment of the computer-based application of the Payor facilitation and health related outcomes tracker.

FIG. 2B illustrates a flow diagram of an example embodiment of the computer-based application of the Payor facilitation and health related outcomes tracker.

FIG. 3 illustrates steps of an example embodiment of the progress tracker

FIG. 4 illustrates a flow diagram of an example embodiment where the patient discovers the computer-based application before meeting with the healthcare provider.

FIG. 5 illustrates a flow diagram of an example embodiment where the computer-based application provides guidance to the patient that may be enhanced by the means of questionnaires.

FIG. 6 illustrates the synergistic effect where efficacy data and higher payor approval rates cause increased use of the device and a friendlier payment environment.

FIG. 7 illustrates an example embodiment of a progress tracker used in the payor approval process.

### DETAILED DESCRIPTION OF THE INVENTION

The present invention is explained in further detail below with references to the drawings.

The components of the present invention are generally described and illustrated in the figures. However, the figures may be arranged and designed in a variety of different configurations. Therefore, the detailed description provided of the embodiments is not intended to limit the scope of the invention as claimed, but only a representation of some embodiments of the invention.

The characteristics or features of the present invention throughout this specification may be combined within any reasonable manner with one or more embodiments. Hence, statements such as “example embodiments”, “example of the embodiment” or other similar language in the specification should be understood to mean that a particular characteristic or feature described concerning an embodiment may be included in at least one possible embodiment of the present invention.

Furthermore, the term “Computer-based application” as described in the specification should be understood to mean many possible software systems including software systems generally found on personal computers as well as mobile software applications for smartphones and tablets. Also, the terms “device” or “drug” are not intended to limit the scope of the invention as claimed because the invention may be used for other medical procedures such as treatments and therapies as well. Also, the term “healthcare provider” refers to the healthcare professional that interacts with the patient such as a medical doctor and the term “medical office” refers to other staff that is usually responsible for handling the patient file.

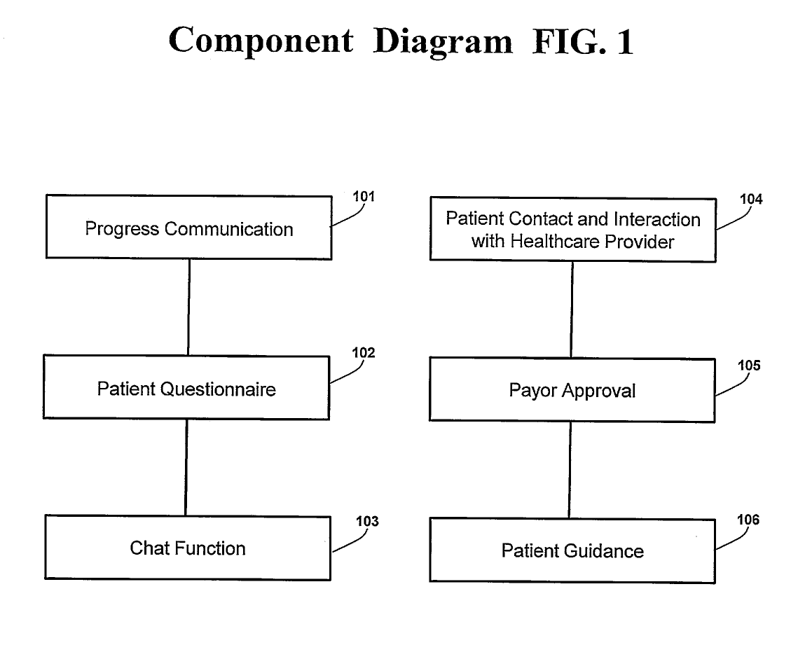

FIG. 1 illustrates a diagram of an example embodiment of multiple components that the computer-based application and health related outcomes tracker may include. Referring to FIG. 1, the computer-based application may have interactive functions 101, 102, 103 as well as functionality features 104, 105, 106. The interactive functions of the computer-based application such as progress communication 101, the patient questionnaire 102 and the chat function 103 aid in keeping the patient engaged in the payor approval process and also provides useful information to the healthcare provider, payors and drug or device manufacturers concerning the patient and the drug or device. The functionality features of the computer-based application such as the patient contact and interaction with the healthcare provider 104, Payor Approval 105 and the Patient guidance 106 help attract and retain users who will be invested in the process.

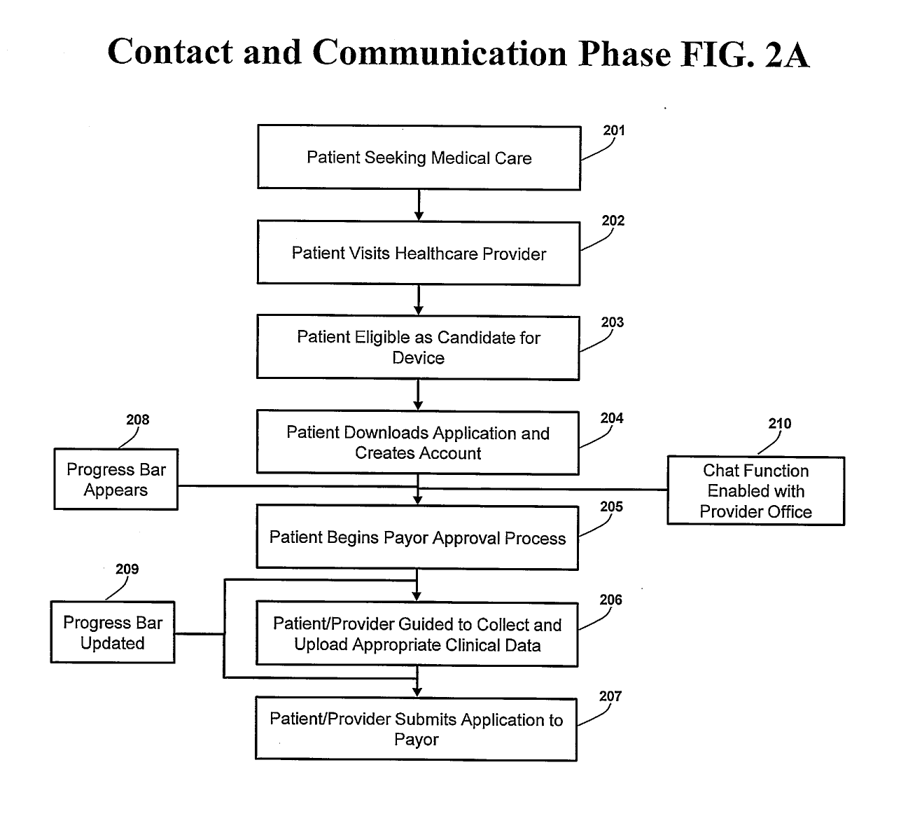

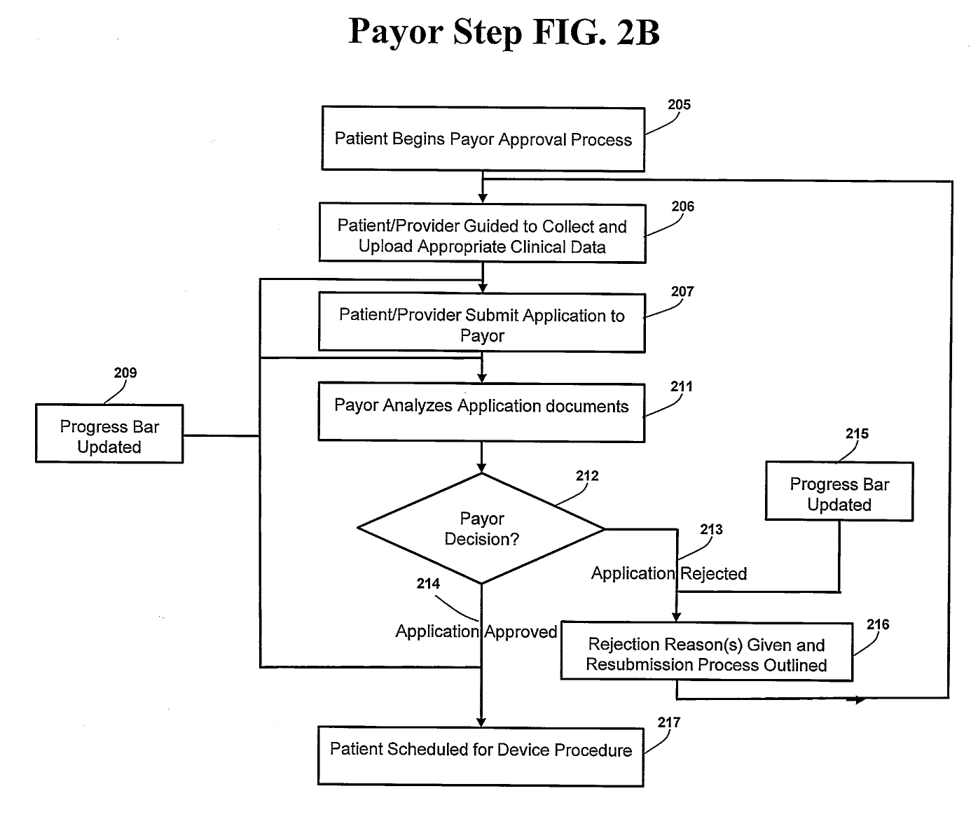

FIG. 2A illustrates a flow diagram of an example embodiment of the Computer-based application of the payor facilitation and health related outcomes tracker. Referring to FIG. 2A, a patient who is a person desiring a drug or device that needs payor approval, usually seeks a healthcare provider 201 or acquires information from the internet about their symptoms. Next, the patient schedules an appointment with the medical office and then visits 202 with a healthcare provider who assesses the patient's condition. If the patient is a suitable candidate 203 for a drug or device requiring insurance pre-approval, the healthcare provider instructs that the patient download the computer-based application and create a personalized patient account 204 on their handheld personal computer with a mobile operating system such as a smartphone. If the patient lacks access to a handheld personal computer with a mobile operating system, another embodiment of the present invention will be available for use on a personal computer.

The downloaded computer-based application will create an individualized progress tracker 208 that will track and update the patient 209 about the progress and status of their patient file as major milestones in the insurance pre-approval process are achieved. Also, another embodiment of the present invention will feature a chat function that allows communication between the medical office and the patient that will be enabled 210 after the computer-based application is downloaded. The chat function 210 allows the patient to interact with their care team thereby keeping them engaged in the process and facilitating efficient communication. It also allows for communication of concerning symptoms. The care team can include, but is not limited, the healthcare provider, nurses, medical assistants, staff, etc.

As the payor approval process 205 begins, the computer-based application guides 206 the patient to collect the appropriate clinical data. The computer-based application will list documents that the patient must upload to create a patient file for submission to get payor approval. To aid the patient, the computer-based application's progress tracker 209 will update after the appropriate clinical data is submitted. In another example of the embodiment, the computer-based application will provide guidance for each specific document that must be provided. In another example of the embodiment, the computer-based application guides 206 the healthcare provider instead of the patient to collect the appropriate clinical data. In another example of the embodiment, both the patient and the healthcare provider will be guided 206 to collect and upload certain documents to complete the patient file.

The type of documents or clinical data that the patient or the healthcare provider uploads 206 to the computer-based application may be, but are not limited to, clinical notes, patient health information, diagnostic testing results, and clinical data supporting the use of the device or drug. The computer-based application can accept documents and clinical data in several potential formats such as, but not limited to, portable document format (pdf), Microsoft Word and jpeg. In an example embodiment, the patient or provider may take screenshots of their documents and upload them. The documents may be uploaded via a document upload feature. In another example of an embodiment, the computer-based application will display the number of documents uploaded 206 and also the number of documents required to be uploaded to complete the file before it is submitted in a visual format along with the progress tracker updates 209.

FIG. 2B continues the illustration of the flow diagram in FIG. 2A that is an example embodiment of the computer-based application of the payor facilitation and health related outcomes tracker. The uploaded documents and clinical data 206 making up the patient file is available to parties facilitating the pre-approval process such as the medical office and/or third-party facilitators facilitating the payor approval process. The patient, medical office and/or third-party facilitators submit 207 the patient file to the payor who reviews and analyzes 211 the patient file submission. The payor will then decide 212 whether to reject 213 the patient pre-approval request or to accept 214 the pre-approval request. In another example of an embodiment, the patient file is directly submitted 207 to the payor though the computer-based application for review.

If the patient file submission is rejected 213 by the payor, the computer-based application will provide reasons for the rejection 216 and guide the patient to a resubmission process 216 that outlines additional documents required to complete the patient resubmission file for the payor pre-approval process. The progress tracker 215 informs the patient about the steps required to complete the patient resubmission file. In another example of an embodiment, the computer-based application will display to the patient the number of documents uploaded and also the number of documents required to complete the patient file resubmission. In another example of an embodiment, the progress tracker's 215 progress indicator such as a progress bar, will update and visually communicate an increase of progress each time a required document is uploaded to the patient file. This decreases the time from payor rejection to resubmission hence decreasing the total time until approval. It also facilitates resubmission which increases the likelihood of eventual approval.

If the patient file is approved 214, the computer-based application informs the patient and the medical office by sending a message. The computer-based application then aids both the medical office and patient in scheduling 217 a timeline for the drug to be dispensed or for the device to be implanted. If the patient requires a device, the computer-based application can schedule 217 the date for surgery. In an example of an embodiment, to facilitate the scheduling 217, the medical office will input a date into the computer-based application. In another example of the embodiment, the patient will have the option of accepting or rejecting the scheduled 217 date. In another example of the embodiment, the computer-based application will display a calendar that allows the patient the option of scheduling the surgery or receive the drug.

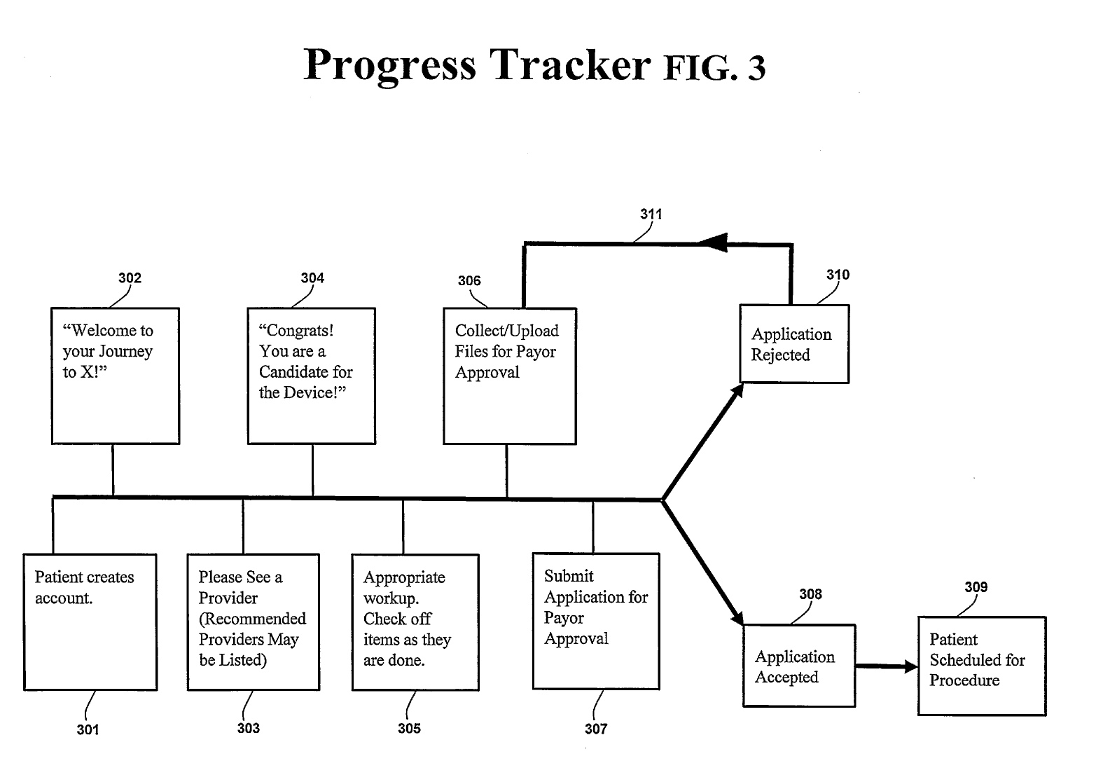

FIG. 3 illustrates a flow diagram of an example embodiment of the progress tracking function of the computer-based application of the payor facilitation and health related outcomes tracker. Referring to FIG. 3, a patient who is a person seeking a drug or a device that needs payor approval will create 301 a personalized account on their handheld personal computer with a mobile operating system. The creation of the personalized account will send 302 a message to patient welcoming the patient to the payor facilitation process. The progress tracker will display a visual representation of the patient's progress by the means of a progress indicator such as, but not limited to, a progress bar that conveys the completion of a task to the patient.

The computer-based application will request 303 that the patient schedule an appointment with a healthcare provider. In another example of an embodiment, the computer-based application will recommend healthcare providers in the patient's vicinity who offer that desired device or drug. In another example of an embodiment of the computer-based application, the patient will be able to schedule an appointment with a healthcare provider using the computer-based application. The progress indicator will convey the completion of the task once the appointment with the healthcare provider has been scheduled. If the patient is a suitable candidate for a drug or device requiring insurance pre-approval, the progress tracker will send 304 a message informing the patient that he or she is a candidate for the drug or device and convey the completion of this threshold to the patient.

The computer-based application will list 305 the documents that the patient must upload to create a file for submission to get payor approval. In another example of the embodiment, the documents that the patient must upload to the patient file will be displayed 305 as a checklist that the patient can interactively check off once the appropriate documents have been collected. The completion of this task will be conveyed to the patient by the progress indicator. The patient is then guided to upload 306 the document for payor approval and the completion of the application submission 307 task is conveyed to the patient by the progress tracker. If the payor accepts 308 and approves the application, the successful completion of the payor approval is conveyed to the patient by the progress tracker and the patient may schedule 309 for the desired drug treatment or device surgical procedure. However, if the payor rejects 310 the application, the progress tracker conveys the unsuccessful result and the computer-based application guides 311 the patient to collect and upload 306 additional documents required for resubmission.

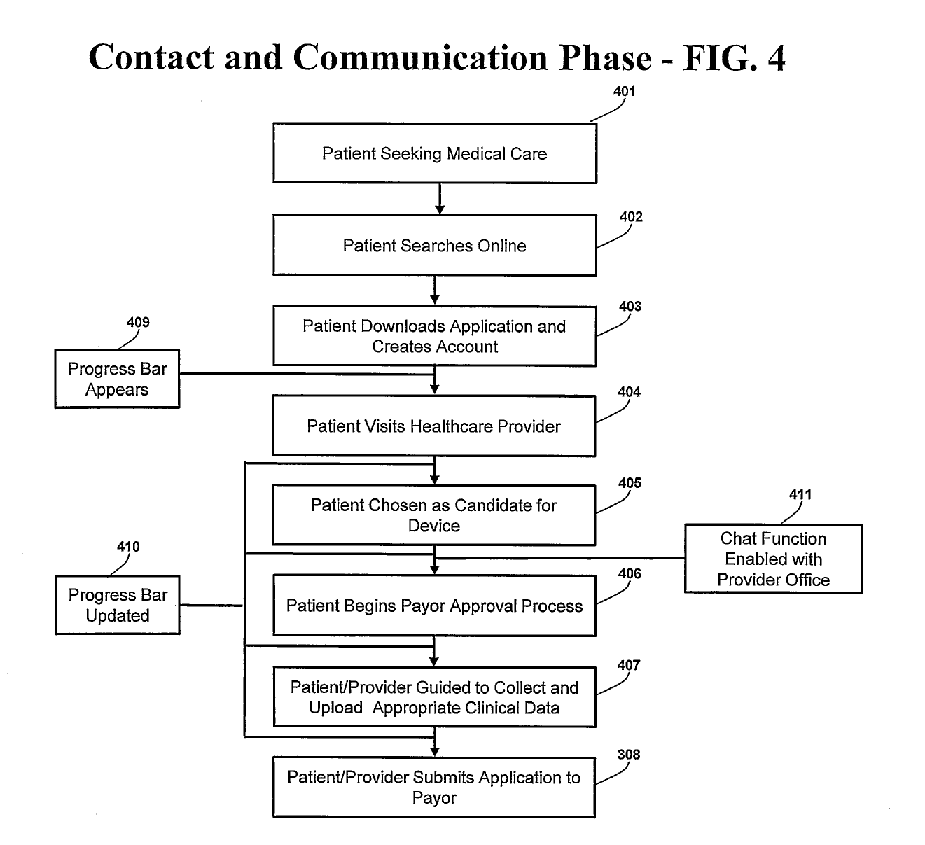

FIG. 4 illustrates a flow diagram of an example of the embodiment of the computer-based application of the payor facilitation and health related outcomes tracker where the patient discovers or is directed to the computer-based application online before scheduling a visit with the medical office and meeting with the healthcare provider. Referring to FIG. 4, a patient who a person desiring a drug or device that needs payor pre-approval, usually seeks a healthcare provider 401 or acquires information from the internet about their symptoms. The patient searches 402 online for their symptoms, treatments for their condition, or for the drug/device specifically. The results of an internet search will guide the patient to an online version of the computer-based application that is available online such as through a web portal and the patient will be instructed to download 403 and create a personalized patient account on their handheld personal computer with a mobile operating system. If the patient lacks access to a handheld personal computer with a mobile operating system, another embodiment of the present invention will be available for use on a personal computer. In another example of an embodiment, the patient's online search 402 leads to a web portal that will describe the drug or device and how the drug or device functions to address the patient's medical condition. In another example of an embodiment, the computer-based application also recommends healthcare providers in the patient's vicinity who offer that drug or device that the patient seeks.

The downloaded computer-based application will create an individualized progress tracker 409 that will track and update 410 the patient about the progress and status of the application by a visual progress indicator like a progress bar as major milestones and events in the payor pre-approval process are achieved. Also, another embodiment of the present invention will feature a chat function that allows communication between the medical office and the patient that is enabled 411 after the computer-based application is downloaded. Much like the progress tracker 410, the chat function 411 gives the patient useful information and engages the patient in the insurance pre-approval process. In an example of the embodiment the chat function 411 will be enabled after the patient downloads 403 the computer-based application allowing the patient to communicate 411 with the medical office. In another example of an embodiment, the enabling of the chat 411 function after the patient downloads 403 the computer-based application will allow the patient to schedule a visit 404 with the healthcare provider through the computer-based application.

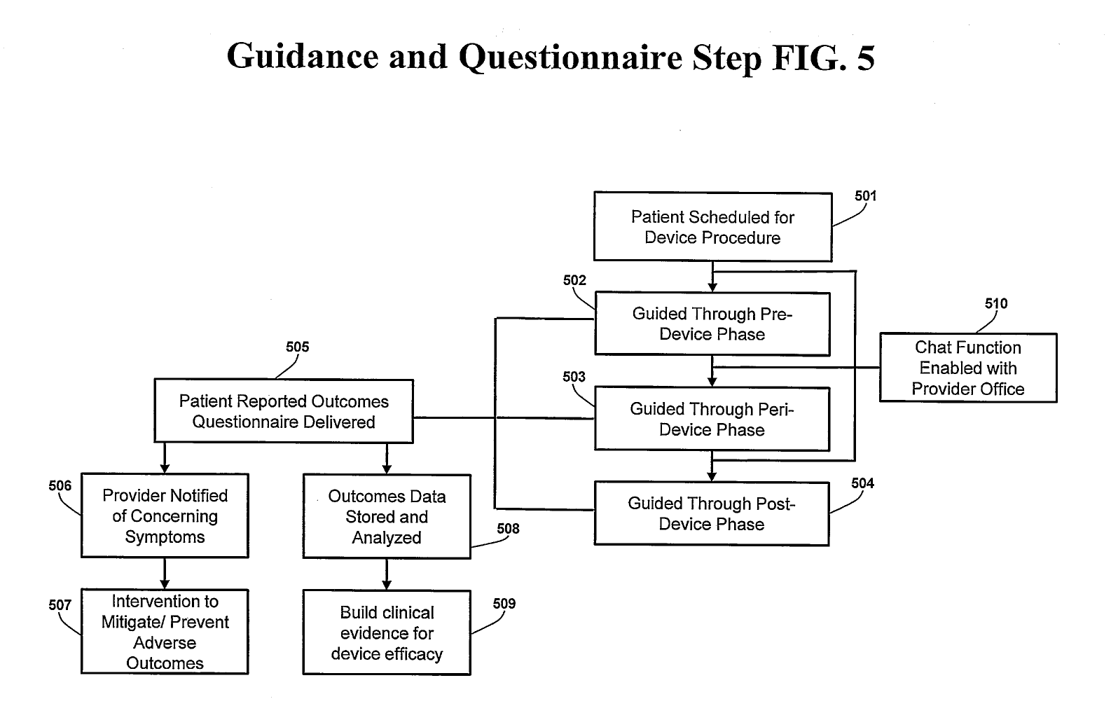

FIG. 5 illustrates a flow diagram of an example embodiment where the computer-based application provides guidance to the patient that may be enhanced by the means of questionnaires. Referring to FIG. 5, the computer-based application of the payor facilitation and health related outcomes tracker provides guidance to the patient who is scheduled 501 for the device procedure. The guidance is provided through the pre-device phase 502 before the surgical procedure, the peri-device phase 503 where the device is inserted and the post-device phase 504 after the device has been inserted. An example of the guidance may be, but is not limited to, the computer-based application displaying a date and time countdown until the drug treatment is completed or until the date of surgery. In another example embodiment, the computer-based application will relay drug or device information to the patient incrementally until the surgery date. It is within the scope of the invention that the patient will not be guided 502, 503, 504 in all the phases. In another example of an embodiment, the chat function 510 will be enabled with the medical office for the patient to ask questions and relay concerns to the medical office.

In another example of an embodiment, the patient will be sent 505 outcomes questionnaires in each of the guidance phases 502, 503, 504. It is within the scope of the invention that the questionnaires will not be sent in all the guidance phases. The patient inputs relevant information into the computer-based application such as symptoms, reactions and pain levels. The patient's inputted information is stored in a database 508 and then relayed to the medical office, payor and/or to the drug or device company who will then build 509 clinical evidence for device efficacy. The post-procedure patient information helps the drug or device producer to gauge efficiency and efficacy of their product. Furthermore, the relevant information that the patient inputs into the computer-based application such as symptoms, reactions and pain levels notify 506 the provider of any concerning symptoms or problems that the patient must address. The provider may then act to mitigate or prevent 507 adverse outcomes during the pre-device 502, peri-device 503, or post-device phase 504. This will also aid in identifying healthcare providers who have better results as well as those who have worse results in order to learn from or improve their performance respectively.

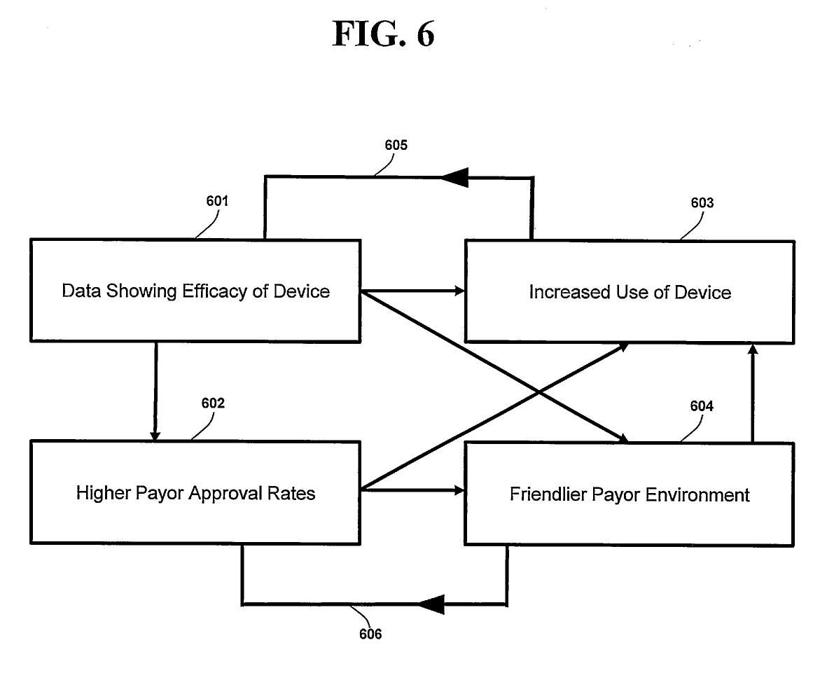

FIG. 6 illustrates the synergistic effect where efficacy data leads to higher payor approval rates which causes increased use of the device and a friendlier payment environment. Referring to FIG. 6, the questionnaires that are sent to the patient will prompt answers that are crucial in gauging the efficacy of a drug or device. This patient provided data can be used to show 601 the efficacy of a drug or device. Data showing 601 drug or device efficacy will lead to higher 602 payor approval rates which will increase 603 the use of the drug or device and also create a friendlier 604 payor environment. The increased 603 use of the drug coupled with a friendlier 604 payor environment will produce more 605 data showing efficacy 601 of the device therefore leading to even higher 602 approval rates which will further increase 603 the use of the drug or device and also create a more friendlier 604 payor environment.

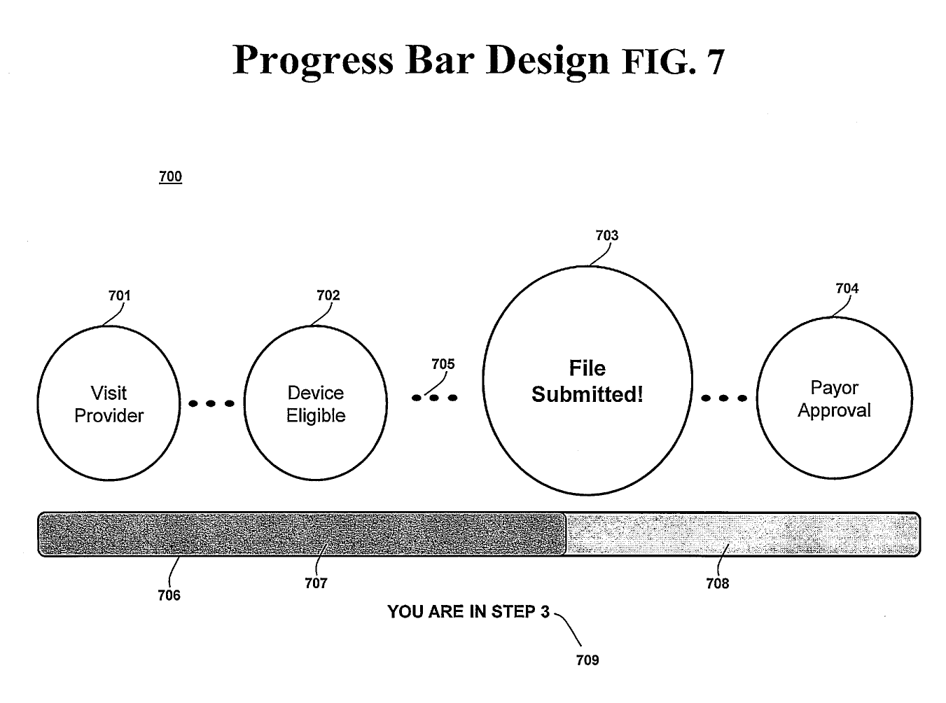

FIG. 7 illustrates an example embodiment of a progress tracker in the payor approval process. Referring to FIG. 7, the progress tracker as seen on a display screen 700 includes a progress bar 708, a current step indicator 709 and circular shapes 701 describing the previous 702, present 703 and future 704 steps or thresholds in the payor approval process. In an embodiment of the invention, the progress bar 706 will change color 707 once a particular step is completed leaving less space uncolored 708. In another example embodiment of the invention, the step that the patient is on will have a circle 703 that will increase in size to help the patient appreciate the progress that has been made and what steps remain 704. In another embodiment of the invention, written language below the progress bar will inform 709 the patient about what step they are on. This will keep the patient informed and invested in the payor pre-approval process.

What is claimed is:

1. A computer-based application for tracking health-related outcomes in an insurance pre-approval process, comprising:
(a) a personalized patient account specific to a user;
(b) an individualized progress tracker for tracking progress of a patient file in the insurance pre-approval process;
(c) a document upload feature permitting the user to upload documents to the patient file.

2. The computer-based application as recited in claim 1, where the individualized progress tracker further provides updates about the progress of a patient file in the insurance pre-approval process.

3. The computer-based application of claim 2, where the updates provided by the individualized progress tracker include major milestones achieved in the insurance pre-approval process.

4. The computer-based application of claim 1, further comprising:
(a) a chat function allowing interaction between the user and a care team.
